(19) United States
(12) Patent Application Publication
(10) Pub. No.: US 2020/0222229 A1
(43) Pub. Date: Jul. 16, 2020

(54) Prophylactic Kit

(71) Applicant: Aasef Shafik, Arlington, VA (US)

(72) Inventor: Aasef Shafik, Arlington, VA (US)

(21) Appl. No.: 16/245,142

(22) Filed: Jan. 10, 2019

(51) Int. Cl. A61F 6/00; A61F 6/04

(52) U.S. Cl. CPC A61F 6/005; A61K 9/0034; A61F 6/04

(57) ABSTRACT

A prophylactic kit has first and second pocket parts foldably joined to each other along opposite sides of a spine and pivotable about the spine from an open position to a closed position. At least one pocket is on the front surface of each pocket part and on the front surface of the spine, including a first pocket on the front surface of one of the pocket parts, a second pocket on the front surface of the other pocket part, a third pocket overlying a portion of the first pocket, and a fourth pocket on the front surface of the spine. The pockets have open upper edges through which items may be inserted into and removed from the pockets. The items include hygienic wipes in the first and second pockets, a condom in the third pocket, and a lubricant in the fourth pocket.

### TECHNICAL FIELD

The present invention relates generally to a prophylactic kit that enhances hygiene and reduces the risk of pregnancy and infection by sexually transmitted disease. More particularly, the kit contains one or more condoms, hygienic male and female wipes, and a lubricant.

### BACKGROUND ART

The practice of safe sex during sexual intercourse is necessary to preserve the health of either partner. A condom is an effective physical barrier for the prevention of both conception and the transmission of sexually transmitted disease. Lubricants are also sometimes used during sexual intercourse.

Recent medical research casts doubt on the effectiveness of the topical application of a microbicide in the vagina or rectum to prevent transmission of disease. Moreover, the results of the research indicate that the use of nonirritating, over-the-counter lubricants should be considered for the inhibition of HIV production. See S. Baron, J. Poast, D. Nguyen, and M. W. Cloyd, “Practical Prevention of Vaginal and Rectal Transmission of HIV by Adapting the Oral Defense: Use of Commercial Lubricants” AIDS Research and Human Retroviruses, Volume 17, Number 11, 2001, pp. 997-1002.)

Most retail pharmacies and many grocery and general retail stores offer hygienic wipes along with vaginal lubricants. with advertising and trade dress targeting the female consumer. These wipes are individually packaged and sold over the counter in bulk quantities and are intended to enhance hygiene and reduce the risk of infection by sexually transmitted diseases.

A condom is designed to cover the penis during sexual intercourse and is worn either for contraceptive purposes or as a barrier for preventing infection of a partner with a sexually transmitted disease, or both. If properly applied and properly removed, the use of a condom works to prevent sexually transmitted diseases during intercourse. A problem arises when there is contact with those surfaces or fluids not protected or blocked by the condom barrier.

There is a large body of art related to condoms, sanitary wipes and lubricants. Prior U.S. Pat. Nos. 4,892,188, 4,974,730, 6,257,400, 6,581,775 6,612,427 and 6,742,521, and published US application 2005/0045497 are exemplary of such devices, but none of them teach the present invention of a kit containing at least one condom, male and female wipes, and a lubricant.

U.S. Pat. Nos. 4,925,033 and 5,244,096 to Stoner disclose a kit for cleaning of the genital area before and after intercourse, principally with povidone-iodine solution. While Stoner discloses the application of povidone-iodine solution with a towelette, he does not disclose a kit that includes at least one condom, hygienic wipes, and a lubricant.

There remains a need for a kit that includes a condom, one or more hygienic wipes, and a lubricant that are all conveniently packaged together in a package that is unobtrusive and easy to carry in a pocket or purse.

### DISCLOSURE OF THE INVENTION

The present invention is a kit that comprises at least one condom, hygienic wipes, and a lubricant, all contained in a package that is unobtrusive and easy to carry in a pocket or purse,

More specifically, the kit of the invention comprises a container or package having first and second pocket parts with front surfaces, back surfaces, top and bottom edges, and opposite end edges. The pocket parts are foldably joined to each other along a spine and are pivotable about the spine from an open position to a closed position with one pocket part overlying the other so that the front surfaces are in confronting juxtaposed relationship. At least one pocket is formed on the front surface of each pocket part and a pocket is formed on the front surface of the spine. The pockets have open upper edges through which items may be inserted into and removed from the pockets. A closure flap may optionally be provided on a top edge of one of the pocket parts so that it can be folded over a top edge of the other pocket part to secure the pocket parts in closed position.

The pockets are defined by front panels adhesively secured in overlying relationship to a back panel. In a preferred construction, a first pocket containing a first hygienic wipe is formed on the front of one of the pocket parts, a second pocket containing a second hygienic wipe is formed on the front of the opposite pocket part, a third pocket containing a condom is formed in overlying relationship to the second pocket, and a fourth pocket containing a lubricant is formed on the front of the spine The upper edges of the second and third pockets are vertically offset from one another and the upper edges of all but the fourth pocket are inclined to provide easier access to the items held in those pockets. When provided, the optional closure flap holds the package closed and prevents loss of product from the pockets during storage and handling of the package.

A blank for erecting the package of the invention comprises first and second side-by-side rectangularly shaped back panels joined to opposite sides of a spine connected between adjacent side edges of the first and second back panels. A first front panel having the shape of a right trapezoid is foldably joined along a first side edge thereof to the first back panel, and a second front panel having the shape of a right trapezoid is foldably joined to the edge of the first front panel opposite the first side edge thereof. The first and second front panels have upper edges that are oppositely inclined, with the upper edge of the second front panel spaced below the upper edge of the first front panel. A third front panel having the shape of a right trapezoid is foldably joined along a first side edge thereof to the second back panel, and a fourth front panel having a rectangular shape is foldably joined to an edge of the third front panel opposite the first side edge thereof. Bands of adhesive along the outer side edges of the first, second, third and fourth front panels hold the front panels in operative position against the back panels.

### BRIEF DESCRIPTION OF THE DRAWINGS

The foregoing, as well as other objects and advantages of the invention, will become apparent from the following detailed description when considered in conjunction with the accompanying drawings, wherein like reference characters designate like parts throughout the several views, and wherein:

FIG. 1 is a front elevation view of the kit of the invention, with the package in open position.

FIG. 2 is a top plan view of the package of FIG. 1, with the package in open position.

FIG. 3 is a bottom plan view of the package of FIG. 1, with the package in closed position.

FIG. 4 is a top isometric view of the package of FIGS. 1-3, with the package in closed position.

FIG. 5 is a plan view of a blank for making the package of FIGS. 1-4.

FIG. 6 is an isometric view of the blank in a partially folded position, illustrating how the front panels are folded onto the back panels to form the pockets.

### BEST MODE FOR CARRYING OUT THE INVENTION

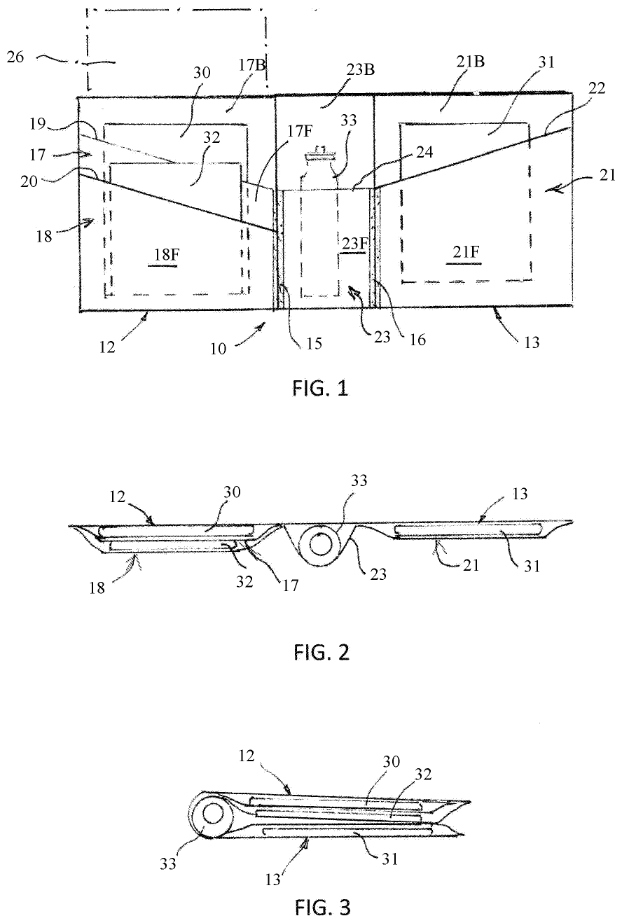

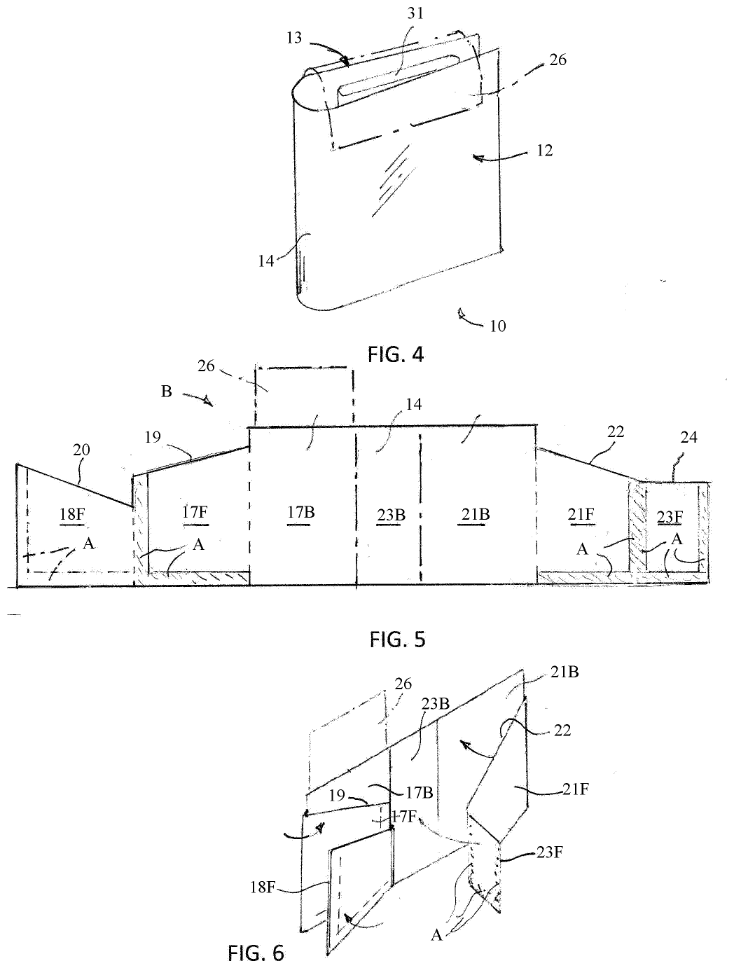

A kit according to the invention is indicated generally at 10 in FIGS. 1-4. The kit comprises a package having first and second pocket parts 12 and 13 joined to respective opposite sides of a spine 14 and foldable about opposite edges 15 and 16 of the spine from an open position as shown in FIGS. 1 and 2 to a closed position as shown in FIGS. 3 and 4, with one pocket part overlying the other so that the front surfaces of the pocket parts are in confronting juxtaposed relationship to one another.

A first pocket 17 is formed on the front surface of the first pocket part 12 and a second pocket 18 overlies a portion of the first pocket. The pockets 17 and 18 extend upwardly from the bottom edge of the pocket part 12 and terminate at angled upper edges 19 and 20, respectively, with the upper edge 20 of the second pocket spaced below the upper edge 19 of the first pocket.

A third pocket 21 is formed on the front surface of the second pocket part 13 and is substantially identical to the first pocket 17, except the upper edge 22 of pocket 21 is angled oppositely to the upper edge 19 of pocket 17.

A fourth pocket 23 is formed on the front of spine 14 and has an upper edge 24 extending between the inner ends of upper edges 19 and 22 of pockets 17 and 21. Edge 24 is substantially parallel to the top and bottom edges of the package.

The upper edges 19, 20, 22 and 24 of the respective pockets are open so that items may be inserted into and removed from the pockets.

A closure flap 26 may optionally be provided on a top edge of one of the pocket parts (pocket part 12 in the example shown) so that it can be folded over a top edge of the other pocket part when the pocket parts are folded into closed position as shown in FIGS. 3 and 4.

The pockets 17, 21 and 23 are respectively defined by front panels 17F, 21F and 23F overlying and secured along their free edges to a respective back panel 17B, 21B and 23B. As seen best in FIGS. 1 and 6, pocket 18 is defined by a front panel 18F overlying and secured along its free edges to the front panel 17F of pocket 17. The edges may be secured by bands of adhesive A or by other suitable fastening means such as, for example, heat bonding.

A blank for erecting the package of the invention is shown at B in FIG. 5. The blank comprises first and second rectangularly shaped back panels 17B and 21B joined to opposite sides of back panel 23B in spine 14. A first front panel 17F having the shape of a right trapezoid is foldably joined along a first side edge to the first back panel 17B, and a second front panel 18F having the shape of a right trapezoid is foldably joined to the front panel 17F along the edge thereof opposite the edge joined to the back panel 17B. The first and second front panels have upper edges 19 and 20, respectively, that are oppositely inclined, with the upper edge 20 of the second front panel 18F spaced below the upper edge 19 of the first front panel. A third front panel 21F having the shape of a right trapezoid is foldably joined along a first side edge thereof to an edge of the second back panel 21B opposite the edge joined to the spine, and a fourth front panel 23F having a rectangular shape is foldably joined to an edge of the third front panel 21F opposite the first side edge thereof.

Bands of adhesive A are along the outer side edges of the first, second, third and fourth front panels to hold the package in closed operative position as described hereinafter.

The blank is folded as shown in FIG. 6 to form the package container of the invention. Thus, with reference to FIGS. 5 and 6, front panel 17F is folded inwardly into overlying relationship to back panel 17B and secured thereto by lines of adhesive A or other suitable fastening means. Panel 18F is then reverse folded into overlying relationship with panel 17F and secured thereto by lines of adhesive A or other suitable fastening means. This forms pockets 17 and 18. Panels 21F and 23F are then folded inwardly so that panel 21F overlies the front of panel 21B and panel 23F overlies the front of the spine 23B. Panels 21F and 23F are then secured in place with the bands of adhesive A or other suitable fastening means, forming pockets 21 and 23.

In a preferred construction, sealed packages 30 and 31 of commercially available hygienic wipes are inserted into pockets 17 and 21 through the slanted open ends thereof. In a preferred embodiment, one of the packages 30 is intended for use by females and may be colored pink and lavender scented, and the other package 31 intended for use by males and colored blue and lemon scented. A sealed package 32 containing a condom is inserted into pocket 18 through the slanted open end thereof, and a dispensing container of lubricant 33 is inserted into pocket 23 through the open upper end 24.

While particular embodiments of the invention have been illustrated and described in detail herein, it should be understood that various changes and modifications may be made in the invention without departing from the spirit and intent of the invention as defined by the appended claims.

What is claimed is:

1. A prophylactic kit comprising:
a package having first and second pocket parts with front surfaces, back surfaces, top and bottom edges, and opposite end edges;
said pocket parts being foldably joined to each other along opposite sides of a spine and pivotable about the spine from an open position to a closed position with one pocket part lying against the other; and
at least one pocket on the front surface of each said pocket part and a pocket on the front surface of the spine, said pockets having open upper edges through which items may be inserted into and removed from the pockets.

2. A prophylactic kit as claimed in claim 1, wherein:
a closure flap is provided on a top edge of one of said pocket parts so that it can be folded over a top edge of the other pocket part to secure the pocket parts in closed position.

3. A prophylactic kit as claimed in claim 1, wherein:
said pockets include a first pocket on the front surface of a first of said pocket parts and a second pocket overlying a portion of the first pocket.

4. A prophylactic kit as claimed in claim 3, wherein:
said pockets further include a third pocket on the front surface of the second pocket part and a fourth pocket on the front surface of said spine.

5. A prophylactic kit as claimed in claim 4, wherein:
said first, second and third pockets have angularly oriented upper edges to enhance access to product contained in the pockets.

6. A prophylactic kit as claimed in claim 5, wherein:
the front surfaces of said pocket parts comprise front panels and the back surfaces comprise back panels, said pockets being formed by said front panels secured along bottom and side edges thereof to said back panels.

7. A prophylactic kit as claimed in claim 6, wherein:
said product comprises hygienic wipes in said first and second pockets, a condom in said third pocket, and a lubricant in said fourth pocket.
